(19) United States
(12) Patent Application Publication
(10) Pub. No.: US 2024/0238105 A1
(43) Pub. Date: Jul. 18, 2024

(54) Improved Prosthesis, Exoskeleton, and Orthosis Devices and Method of Manufacturing Thereof

(71) Applicant: VRIJE UNIVERSITEIT BRUSSEL, Elsene (BE)

(72) Inventors: Joost GEEROMS, Elsene (BE); Louis FLYNN, Elsene (BE); Dirk LEFEBER, Elsene (BE)

(21) Appl. No.: 18/289,824

(22) PCT Filed: May 11, 2022
(86) PCT No.: PCT/EP2022/062736

§ 371 Date: Nov. 7, 2023

(30) Foreign Application Priority Data
May 11, 2021 (EP) 21173331.6

(51) Int. Cl. A61F 2/64; A61F 2/30; A61F 2/50; A61F 2/70; A61F 5/01

(52) U.S. Cl. CPC A61F 2/64; A61F 2/70; A61F 5/0102; A61F 2002/30471; A61F 2002/5007; A61F 2002/701; A61F 2005/0155

(57) ABSTRACT

The current invention relates to a device for replacing or augmenting a limb of a user, having a joint for connecting an upper and lower part of the assistive device, the joint having an axis of rotation. The device further comprises a joint, an actuator linkage, a base structure, and a linkage mechanism comprising at least three linking elements, wherein a first end of the linkage mechanism is coupled to the joint, a second end of the linkage mechanism is coupled to the actuator linkage, and a third end of the linkage mechanism is coupled to the base structure, wherein the actuator linkage applies a torque to the joint through linkage mechanism.

### FIELD OF THE INVENTION

The present invention relates to a prosthetic device. In particular, the invention relates to a prosthetic device for replacing or augmenting a limb of a user.

### BACKGROUND

An above-knee amputation is a highly debilitating condition. Transfemoral amputees exert as much as twice the energy of their counterparts with fully intact lower limbs when walking on level ground. Tasks such as climbing stairs or standing up from a seated position are exceedingly more difficult for transfemoral amputees.

The research on powered lower limb prosthetics has been focusing on various ways of increasing energy efficiency and reducing weight and size. Nevertheless, few active prosthesis concepts make it to commercial products, which is linked to limited reimbursements and high price of robotic prostheses, but also to the lasting difference in mass between active and passive devices.

Existing commercially available knee prostheses are almost exclusively passive devices. This can be either simple mechanical systems or more advanced adjustable damping devices. Even though these devices cannot be used to inject energy into the gait cycle like able-bodied individuals are capable of doing by means of muscle contractions, they can perform reasonably well when approximating ground level walking. This is because a human knee dissipates energy during walking, mostly slowing the leg down during the swing phase. Transfemoral (above-knee) amputees still show higher metabolic cost than able-bodied individuals, and any other task besides walking, such as slope or stair ambulation or sit-to-stand transfer, require significant power to be generated in the knee joint. Indeed, standing up from a seated position is a relevant clinical problem for all lower limb amputees. Individuals classified as KO-2 on the Medicare Functional Classification Level experience the sit-to-stand transfer as a key factor for leading an independent lifestyle. It has already been shown that transitioning from passive to microprocessor controlled knees can cause low mobility amputees to spend less time sitting and increase their activity level.

Most existing robotic knee joints, either research prototypes or the scarce commercial ones are targeting highly active individuals (K3-4), providing a leg prosthesis that is capable of outputting a behavior that is similar to the capabilities of a healthy leg. With these higher capabilities also comes the need for advanced and complex detection and control algorithms to enable a proper use of this added power. These active prosthetic modules, being heavy and larger than the passive alternatives, might mismatch with the needs and requirements of the low activity-level transfemoral amputees who cannot deal with high prosthesis weight yet could use the added power. Many prosthetic and exoskeleton actuators attempt to include elasticity and specific mechanical layouts to optimize the use of motor power in assisting or replacing a knee actuator. In the agonist-antagonist active knee prosthesis two series elastic actuators act in a different direction of the knee joint, allowing to adjust both the torque and the stiffness of the actuated joint. In the CYBERLEGs Gamma prosthesis a ball-screw mechanism is used to control a push-pull rod acting on the knee joint lever arm. A similar mechanism comprising a ball-screw driving a rod that actuates a lever arm has been used in a knee exoskeleton to assist in sit-to-stand motions. A knee exoskeleton also comprising a ball-screw driven actuation principle yet using a lever arm compressing a linear spring through a cable system also investigated the effect of adjustable compliance. All of these actuators have in common that they struggle with approximating a healthy knee joint behavior by means of only one motor. The reason for this is that during walking a healthy knee joint provides a high stance torque and a high (ground clearing) velocity at low knee angles. If stair climbing or assistance during sit to stand are considered, then the actuator must also provide a high joint torque at high knee angle. Often a compromise needs to be made between the included motor power (and thus weight) and the performance (possible walking velocity and maximum torque). To solve this duality in required knee behavior, Lenzi et al. (“Actively variable transmission for robotic knee prostheses,” in 2017 IEEE International Conference on Robotics and Automation (ICRA). IEEE, 2017, pp. 6665-6671) implemented a second motor to adjust the gear ratio of the actuator when necessary, allowing users to both walk and perform stair ambulation requiring a limited motor power.

Passive prosthetic knees provide some support for walking. Some passive prosthetic knees incorporate microprocessor devices to intelligently control the compliance of the knee. A passive prosthetic knee does not provide additional power to the knee beyond the power provided by its user. A user of a passive prosthetic knee must compensate to adapt to the lost knee power during walking and while performing other tasks. For instance, a user of a passive prosthetic knee may lead with his or her sound limb when climbing curbs or stairs. As another example, users of passive prosthetic knees will “side step” up or down ramps. Some users of passive prosthetic knees do not have the strength to perform these tasks by compensating for the lack of power from the knee. In particular, elderly transfemoral amputees generally do not have the strength to use a passive prosthetic knee. As a result, many elderly transfemoral amputees, particularly those without family support, live in nursing homes rather than their own homes.

A powered prosthetic knee can provide a user with lost functionality by providing power similar to power provided by a biological knee. One commercially available powered prosthetic knee is the Power Knee by Ossur (Reykjavik, Iceland), which uses a motor to provide the power. A powered prosthetic knee can give more functionality to its user. However, in the prior art, the added functionality comes at the cost of a prosthetic knee with greater weight. For example, the Power Knee weighs 3.19 kg (7.1 lbs), more than twice that of most passive prosthetic knees. The extra weight in a powered prosthetic knee is in the motor, transmission, and battery needed to provide sufficient power over a reasonable period of time. The user must carry this extra weight when walking, climbing stairs, or performing other tasks.

US20166/0158029 describes systems and methods for assistive devices for replacing or augmenting the limb of an individual, such devices comprising a joint and a powered system; the powered system having a first configuration in which the powered system rotates the joint by applying power to the joint, and a second configuration that allows for rotation of the joint without actuation of the powered system.

CN112206079 describes an active and passive bionic artificial limb knee joint, which comprises a base, a damping cylinder, a front connecting rod, a receiving cavity connecting piece, an upper connecting rod seat, two rear connecting rods, two telescopic rod assemblies, a ball screw assembly, a linear guide rail assembly, a supporting seat, a synchronous belt assembly, a shank connecting piece and a motor, wherein the telescopic rod structure controlled by a steering engine is adopted for switching the active mode and the passive mode.

JP4194150 describes a prosthesis including a knee joint having a multi-joint link, and more particularly, to a prosthesis including a knee joint having a configuration in which a main four-node limited chain is rotatably connected to a lower leg member at one position.

U.S. Pat. No. 5,545,232 describes a device for mutual pivoting connection of parts of an orthopaedic apparatus, such as in particular a knee prosthesis for leg amputees, comprising a kinematic multiple linkage system with at least four rods, adjoining rods of which have a common pivot axis and the pivot axes extend substantially mutually parallel.

To date, powered prostheses have not improved the efficiency of gait. We believe this is due to the extra weight of powered prostheses in the prior art, including weight from the motor, transmission, and battery. Powered lower limb prostheses improve a user's ability to climb stairs, but most people do not climb stairs during much of the day. The ability to climb stairs and perform other tasks that require a powered prosthetic knee is important to accomplish many activities of daily living, but such tasks make up a relatively small portion of a user's daily mobility needs. There is need therefore to improve the performance of prostheses during stair climbing and standing up from sitting.

The present invention aims to resolve at least some of the problems and disadvantages mentioned above.

### SUMMARY OF THE INVENTION

The present invention and embodiments thereof serve to provide a solution to one or more of above-mentioned disadvantages. To this end, the present invention relates to a prosthetic device for replacing or augmenting a limb of a user, according to claim 1.

The device comprises:
a joint,
an actuator linkage,
a base structure, and
a linkage mechanism comprising at least three linking elements,
   wherein a first end of the mechanism is coupled to the joint, a second end of the mechanism is coupled to the actuator linkage, and a third end of the mechanism is coupled to the base structure, characterized in that the actuator linkage applies a torque to the joint through the linkage mechanism.

The torque of the actuator linkage may be tunable in a range between a predetermined high torque value and a predetermined low torque value.

The high torque value may be at a high-joint angles, and the low torque value may be at low joint angles.

The actuator linkage applies the torque by driving at least one linking element.

The elements in the mechanism may be pivotally coupled.

The mechanism may be rotationally coupled to the joint and the actuator linkage.

The actuator linkage may comprise a linear actuator. For example, the actuator may comprise a motor, a transmission, and a screw. The screw may be, for example, a leadscrew, a ballscrew, or a rollerscrew.

The actuator linkage may comprise a motor for actuating the linear actuator, the linear actuator being positioned so that the actuating axis of the linear actuator is parallel to the axis of the motor.

The device may further comprise a crank member having a first end attached to the actuator linkage and a second end connected to the linkage mechanism. For example, to one of the linking elements.

The device may be a prosthesis and/or an orthosis, such as an upper-limb prosthesis/orthosis, a lower-limb prosthesis/orthosis, such as a knee prosthesis/orthosis.

Preferred embodiments of the device are shown in any of the claims 2 to 9.

It is an advantage of embodiments of the present invention that the device assists a user during walking and sit-to-stand tasks.

It is an advantage of embodiments of the present invention that the performance of prostheses during stair climbing and standing up from sitting is improved, without needing all the power associated with approximating a healthy human knee. It is an advantage of embodiments of the present invention that a low power prosthetic device is obtained. It is an advantage of embodiments of the present invention that the prosthesis can replace a healthy human knee in all normal human movement, especially climbing stairs and standing up from sitting.

It is an advantage of embodiments of the present invention that the assistive device comprises a lightweight, lower-limb prosthesis.

It is an advantage of embodiments of the present invention that the device assists a user during walking and sit-to-stand tasks.

It is an advantage of embodiments of the present invention that the disclosed mechanism described herein allows for an increased performance with respect to passive devices at a limited extra weight. The design presents a trade-off between capabilities and performance and does not try to reproduce the full range of power capability as a healthy human leg, yet attempts to fulfil selected important tasks in an optimal way.

It is an advantage of embodiments of the present invention that the device described herein provides high joint velocities (120 rpm) for walking and high torques (+80 Nm) for sit-to-stand while being very limited in weight (2.3 kg for a fully integrated and autonomous device). The device's performance has been quantitatively verified on a test bench setup as well as during a pilot amputee experiment, showing the capability to perform walking at normal walking velocities with an assisted swing phase and reducing muscle activity during a sit-to-stand task.

The actuator e.g. knee actuator disclosed herein attempts to solve the problem of the high requirements when considering both walking and assisting during sit-to-stand tasks and stair ambulation. This is done by implementing a novel mechanism that allows to tune the transmission ratio from the motor to the output joint in such a way that the actuator can provide high velocities at low knee angles and high torques at high knee angles. The designed four-bar mechanism can allow for a full range at the knee joint from 0° to 120°, and the transmission ratio in this range is shaped as desired. The singular positions of the mechanism corresponding to a zero-transmission ratio and an infinite one, can be placed just outside of this range. The prosthetic knee module including all electronics, sensors and batteries weighs only 2.3 kg and allow for walking at normal walking velocities being able to control the knee joint up to 120 rpm as well as providing torques higher than 80 Nm. The main target group for this device are elderly people and lower mobility amputees, as they can benefit most from having the fast and accurate ground clearance as well as from the assistance when getting up or taking stairs.

In a second aspect, the present invention relates to a method comprising the steps of providing a joint, providing an actuator linkage, providing a base structure, providing a linkage mechanism comprising at least three linking elements, linking the joint, the actuator linkage, and the base structure via the linkage mechanism, and adapting the actuator linkage to apply a torque on the linkage mechanism.

The method may comprise the step of tuning the torque between a high torque value for high joint angles, and low torque value for low joint angles.

The method may comprise the step of adapting the mechanism to rotationally couple to the joint and to the actuator.

The method may comprise the step of providing a linear actuator and providing a motor to the actuator linkage.

In a third aspect the present invention relates to a use of the device as a prosthesis and/or orthosis device for replacing or augmenting a limb of a user according to claims 1 to 12 and/or a method according to the invention.

### BRIEF DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

FIG. 1-2 schematically presents the implementation of the invention in a knee prosthesis.

FIG. 3 shows the knee prosthesis prototype, comprising an active ankle joint and an active knee joint module, according to embodiments of the present invention.

FIGS. 4A and 4B show a schematic representation of the XLeg layout, according to embodiments of the present invention.

FIG. 5 shows a schematic representation of the layout of the CYBERLEGs Gamma prosthesis actuation, according to prior art.

FIG. 6 shows a typical transmission ratio of the implemented four-bar mechanism, according to embodiments of the present invention.

FIG. 7 shows a X-Leg knee prosthesis CAD. The actuator including the four-bar transmission mechanism and the motor-gearbox combination are shown, according to embodiments of the present invention.

FIG. 8 shows a X-Leg knee prosthesis CAD. The covers and the placement of the electronics on the side of the actuator are shown, according to embodiments of the present invention.

FIG. 9 shows a state chart controller for the walking task, according to embodiments of the present invention.

FIG. 10 shows a state chart controller for the Sit-to-stand task.

FIG. 11 shows a preliminary amputee walking experiment used to tune the behavior of the prosthesis.

FIG. 12-13 shows a sit-to-stand experiment performed with both the own prosthesis and the X-leg knee prosthesis.

FIG. 14 shows the transmission ratio of the prosthetic actuator over the entire range of 0-120° of knee flexion.

FIG. 15 shows a torque range for the knee prosthesis for different knee angles.

FIG. 16 shows prosthetic Knee angles during the 2-minute walking experiment.

FIG. 17 shows a comparison between knee joint angle and angular velocity during walking

FIG. 18 shows average integrated muscle activity of the m. tibialis anterior (TA NAMP), m. vastus lateralis (VL NAMP), m. soleus (SOL NAMP), m. gastrocnemius medial head (GAS NAMP), m. vastus medialis (VM NAMP), m. rectus femoris (RF NAMP), m. biceps femoris caput longum (BF NAMP), m. gluteus maximus (GLUT NAMP) and the m. gluteus maximus (GLUT NAMP) on the amputated side (GLUT AMP) during the walking task. The difference in muscle activity (% of maximum voluntary contraction) between the current prosthesis (MAUCH) and novel prosthesis (X-Leg knee) is shown.

FIG. 19 shows ground reaction force measurements during the sit-to-stand experiment using the test subject's own prosthesis.

FIG. 20 shows GRF measurements during the Sit-to-stand experiment using the novel X-Leg knee prosthesis.

FIG. 21 shows average integrated muscle activity of the m. tibialis anterior (TA NAMP), m. vastus lateralis (VL NAMP), m. soleus (SOL NAMP), m. gastrocnemius medial head (GAS NAMP), m. vastus medialis (VM NAMP), m. rectus femoris (RF NAMP), m. biceps femoris caput longum (BF NAMP), m. gluteus maximus (GLUT NAMP) and the m. gluteus maximus (GLUT NAMP) on the amputated side (GLUT AMP) during the sit-to-stand task. Vertical lines indicate one standard deviation across different repetitions.

### DETAILED DESCRIPTION OF THE INVENTION

The present invention concerns a device for replacing or augmenting a limb of a user.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far as such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g., component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination. In a first aspect, the invention relates to a prosthetic device for replacing or augmenting a limb of a user.

The device comprises a joint, an actuator linkage, a base structure, and a linkage mechanism comprising at least three linking elements. A first end of the mechanism is coupled to the joint, a second end of the linkage mechanism is coupled to the actuator linkage, and a third end of the linkage mechanism is coupled to the base structure,
characterized in that the actuator linkage applies a torque to the joint through the linkage mechanism. For example, by pushing the linkage mechanism, which consequently pushes the joint.

The first of the three elements may be coupled to the joint and to the second linking element, the second linking element may be coupled to the actuator linkage and to the first linking element, and the third linking element may be coupled to the second linking element and to the base structure (e.g., grounded structure).

In a preferred embodiment, the device comprises a knee structure, rotatably attached to the base structure via the joint. The actuator linkage comprises a linear actuator with a nut and a motor to actuate the linear actuator, wherein the actuator linkage connects a motor joint and a nut joint. A first of the linking elements is a push/pull link and connects the nut joint and a knee lever arm joint, said push/pull link comprising a structural flexible element and being elastic, wherein the knee lever arm joint is positioned on the knee structure. A second of the linking elements connects the nut joint to the base structure. A third of the linking elements consists of a lever arm link, which connects the knee joint and the knee lever arm joint. It is to be understood that the nut joint and nut are connected, preferably in that the nut joint forms part of the nut, or is directly attached thereto.

The term “nut” is used in a non-restrictive manner, and refers to a part of the linear actuator that is moved relative to other parts of the linear actuator (motor, motor joint, etc.), for instance by a linear actuator link.

In a preferred embodiment, the linear actuator is of a ball screw type, comprising a ball screw, wherein the nut is a ball screw nut, and is moved by the ball screw.

In an alternative embodiment, the linear actuator is of a leadscrew type, comprising a leadscrew, wherein the nut is a leadscrew nut, and is moved by the leadscrew.

In a preferred embodiment, the linear actuator is of a roller screw type, comprising a roller screw, wherein the nut is a roller screw nut, and is moved by the roller screw.

In a possible embodiment, the linear actuator is a linear motor which directly moves the nut.

In a preferred embodiment, the torque of the actuator linkage is tunable in a range between a predetermined high torque value and a predetermined low torque value.

In a preferred embodiment, the high torque value is at high joint angles, and the low torque value is at low joint angles.

In a preferred embodiment, the actuator linkage applies the torque by driving at least one linking element.

In a preferred embodiment, the elements in the mechanism may be pivotally coupled.

In a preferred embodiment, the mechanism may be rotationally coupled to the joint and the actuator.

In a preferred embodiment, the actuator may comprise a linear actuator. For example, the actuator may comprise a motor, a transmission, and a screw. The screw may be, for example, a leadscrew, a ballscrew, or a rollerscrew.

In a preferred embodiment, the device may comprise a motor for actuating the linear actuator, the linear actuator being positioned so that the actuating axis of the linear actuator is parallel to the axis of the motor. The linear actuator may comprise a rotating electrical motor, which is coupled to a linear screw-nut system. The actuator may convert rotating motion to linear motion, and vice versa. In another embodiment, a roller screw and a roller nut may be employed.

In a preferred embodiment, the device may further comprise a crank member having a first end attached to the actuator and a second end connected to the linkage mechanism. For example, to one of the linking elements. For example, the linkage mechanism and the actuator form a five-bar mechanism.

In a preferred embodiment, the device may be a prosthesis and/or orthosis device, such as an upper-limb prosthesis/orthosis, a lower-limb prosthesis/orthosis, such as a knee prosthesis/orthosis.

In a preferred embodiment, the assistive device comprises three linking elements. The first element and the second element may be connected by a joint structure. For example, the joint structure may comprise a plurality of joints that connect the first element and the second element. The second element and the third element may be connected by a joint structure. Each element may comprise a one or more element to form a linkage. Any of the linkages or the joints may be flexible. The linkage mechanism may comprise a clutch on any of the three elements. The mechanism comprising the three elements is referred to as the linkage mechanism.

In a preferred embodiment, the linkages in the polycentric mechanism may be proportioned in order to provide the mechanism with an angular range of motion that mimics the angular range of motion of a natural, human knee. For example, for a knee prosthesis, a knee range of motion for typical mobility covers from 0° in flexion to 120° in flexion. A knee position of 0° flexion is the position of the knee where the upper leg is in line with the lower leg. A flexed knee position is the position where the heel is moved closer to the upper leg.

In various embodiments described herein, the knee range of motion covers from at least 0° to at least 90° in flexion. Additionally, certain linkages may be proportioned in order to provide a prospected displacement of the linkage mechanism. For instance, when the knee angle is close to 0°, the linkage mechanism is proportioned in such a way that the actuator linkage can exert the velocities that would otherwise be provided by a natural knee during walking or running. When the knee angle is close to 120°, the linkage mechanism is proportioned in such a way that the actuator linkage can exert the torques that would otherwise be provided by a natural knee during stair climbing.

In a second aspect, the invention relates to a method for replacing or augmenting a limb of a user. The method comprises the steps of providing a joint, providing an actuator linkage, providing a base structure, providing a linkage mechanism comprising at least three linking elements, linking the joint, the actuator linkage, and the base structure via the linkage mechanism, and adapting the actuator linkage to apply a torque to the joint through the linkage mechanism.

In a preferred embodiment, the nut is movable along the actuator linkage free from the base structure.

In a preferred embodiment, the method further comprises the step of tuning the torque between a high torque value for high joint angles, and low torque value for low joint angles.

In a preferred embodiment, the method further comprises the step adapting the mechanism to rotationally couple to the joint and to the actuator.

In a preferred embodiment, the method further comprises the step of providing a linear actuator and providing a motor to the actuator linkage.

In a third aspect, the invention relates to a use of the device of the first aspect as a prosthesis and/or orthosis device for replacing or augmenting a limb of a user, and/or a method of the second aspect.

The invention is further described by the following non-limiting examples which further illustrate the invention, and are not intended to, nor should they be interpreted to, limit the scope of the invention.

The present invention will be now described in more details, referring to examples that are not limitative.

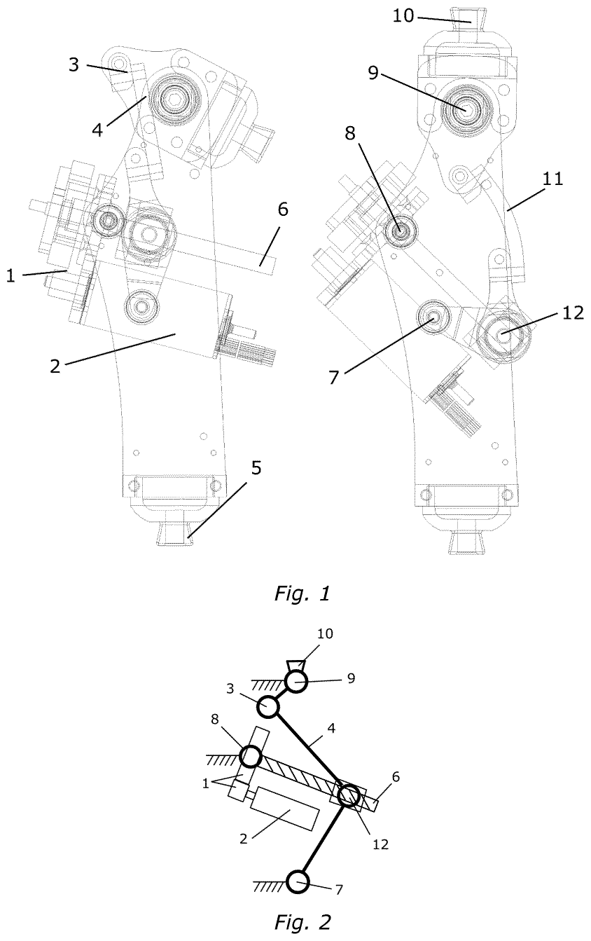

With as a goal illustrating better the properties of the invention the following presents, as an example and limiting in no way other potential applications, a description of a number of preferred applications of the method for examining the state of the grout used in a mechanical connection based on the invention, wherein: As shown in FIG. 1-2, a linear actuator is used, in this case constructed by means of an electric motor (2) and spur gearbox (1) in combination with a ball-screw (6). This actuator is connected to the main structure by means of a hinge (8). A four-bar mechanism is used to transmit the force in the ball-screw and apply a torque to the knee joint (9). The four bars in the actuation unit are the following: between point (9) and point (3), between point (3) and point (12), between point (12) and point (7), and the actuator linkage between point (12) and point (8). By actuating the motor and ball-screw, the length of the link between points (12) and point (8) can be changed, which changes the angle of the knee joint. The link between points (3) and point (12) can be used as an elastic element such as (4) or (11), making the actuation unit a series elastic actuator which can be controlled in torque by measuring the deflection of the link between (12) and (3). This can be done by measuring any 2 of the 4 hinge angles (7,8,9,12) or one of these hinge angles and the motor position, or any other linear or angular position of the mechanism which can indicate the deformation of the four-bar mechanism.

FIG. 2 also shows that the motor joint and the base joint are connected to the base structure and thus are not movable with respect to each other.

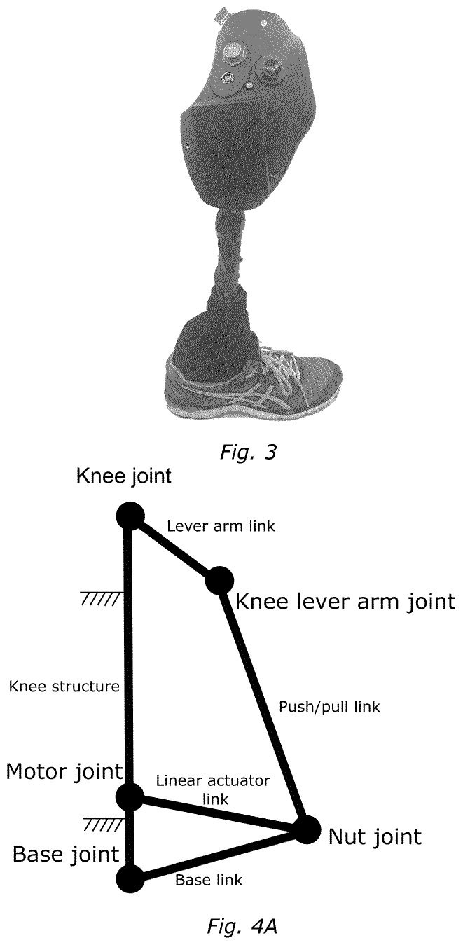

As shown in FIG. 3, the knee prosthesis is entirely self-contained, including the actuation mechanism, all the required sensors, control electronics and a battery pack for normal operation. The module including all these components only weighs 2.3 kg.

The active transfemoral prosthesis consists of a novel active knee actuator and a commercially available passive ankle actuator. The system can be used as part of the CYBERLEGs++ ortho-prosthetic system, thus can be interfaced with an active ankle prosthesis, a Wireless Sensory Apparatus as well as the other orthotic modules developed in the CYBERLEGs++ project. The entire prosthetic leg, so including passive ankle module, 500 g battery pack, tubing and adaptors and adjusted for the test subject's height and preferences, weighed 3.18 kg. For reference, the own prosthesis of the test subject (consisting of a MAUCH knee (Ossur, Iceland), passive foot and cosmetic covers) weighed 2.81 kg.

The prosthesis consists of a Maxon 200W 24V EC-4Pole motor (Maxon 305013) with a 1024 cpt 16 EASY XT incremental encoder (Maxon 530965). The power comes from eight Samsung Lithium-Ion 3.7 Volt batteries (ICR1865026F) in series, providing 29.6V nominally, which are mounted to the side of the prosthesis. There is an absolute encoder (AMS AS5047P) measuring the knee joint angle and another measuring the angle of the actuator carriage. The gearbox is a one-stage gearbox with a 3:1 gear ratio, connected to an 8 mm ball screw with a 2 mm lead.

As shown in FIGS. 4A and 4B, the knee joint and motor joint are connected through the knee structure. The motor drives the linear actuator link, in the case of FIG. 4B a ball-screw link, which moves the (ball-screw) nut joint. This causes a force in the push-pull link and on the knee lever arm joint, which results in a torque around the knee joint.

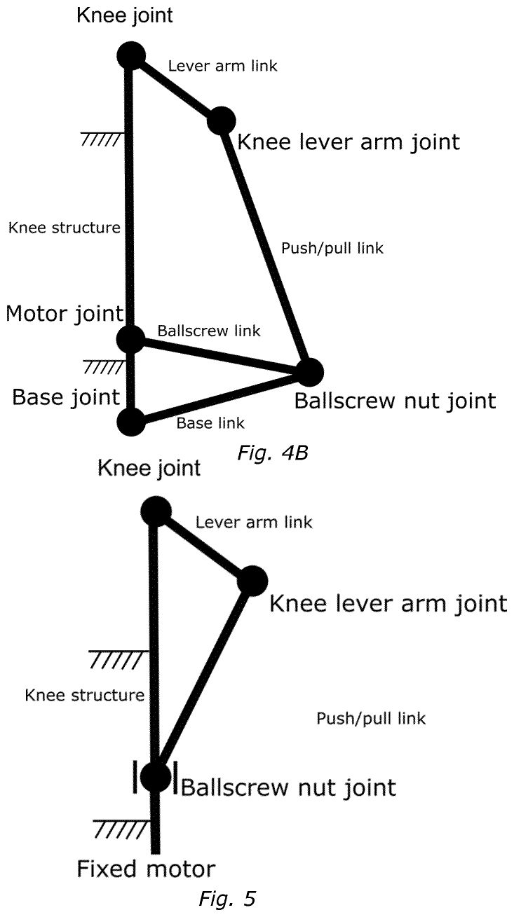

The actuator consists of a four-bar mechanism (i.e. one actuator linkage and three linking elements), connecting the motor with the prosthetic knee joint. The four links are the following: one actuator linkage connecting the motor joint and the ball-screw nut joint, one link connecting the ball-screw nut joint and the knee joint lever arm joint (the push/pull link), one link connecting the ball-screw nut joint to the base and the final link consisting of the lever arm link. The push/pull link is in this case made elastic by incorporating a structural flexible element. A schematic drawing of the concept can be seen in FIG. 4A and FIG. 4B. The specific layout of this actuator has some interesting conceptual differences with state-of-the-art ball-screw-driven actuators. It can easily allow for a range of 120°, while having the maximum torque not in the middle of the range but rather at the edge. Other ball-screw mechanisms incorporate a sinusoidal behavior inherent to their conceptual design. This implies that if one would want to provide the highest torque at highest knee angles, this should correspond to the largest lever arm length and thus the range of the knee joint should be limited. Vice-versa, if the full range of the knee joint is to be utilized, this implies that the maximal torque will decrease again going towards the end of the range. The actuators in the examples above are implemented through a three-bar mechanism, where the length of one of the links is changed by means of the ball-screw mechanism as can be seen in FIG. 5.

The base structure is connected to e.g., an extension leg, as shown in FIGS. 3 and 4.

As shown in FIG. 5, the motor drives the ballscrew nut joint, which pushes against the lever arm link to create a knee torque, as described in prior art.

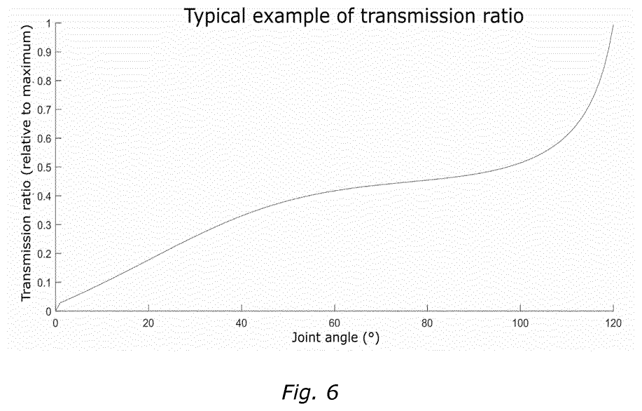

The exact shape of the transmission profile can be adjusted and optimized by tuning the lengths and positions of the different links and joints of the four-bar mechanism. While this can be the subject of an entire investigation by itself, the parameters for the prosthesis actuator were chosen in such a way that they result in a reasonable range for the ball-screw transmission, accommodate the range of motion of the knee joint, and have the desired shift from low to high transmission ratio as the knee angle increases. FIG. 6 shows what this profile typically can look like, however even within the boundary conditions indicated here, there are many possibilities to trade-off between velocity and torque, especially in the center of the range between 30° and 90°. As shown in FIG. 6, the graph illustrates the low transmission ratio at low knee angles and increasing transmission ratio for higher knee angles. The specific shape of the transmission profile can be adjusted and tuned by changing link lengths and joint positions.

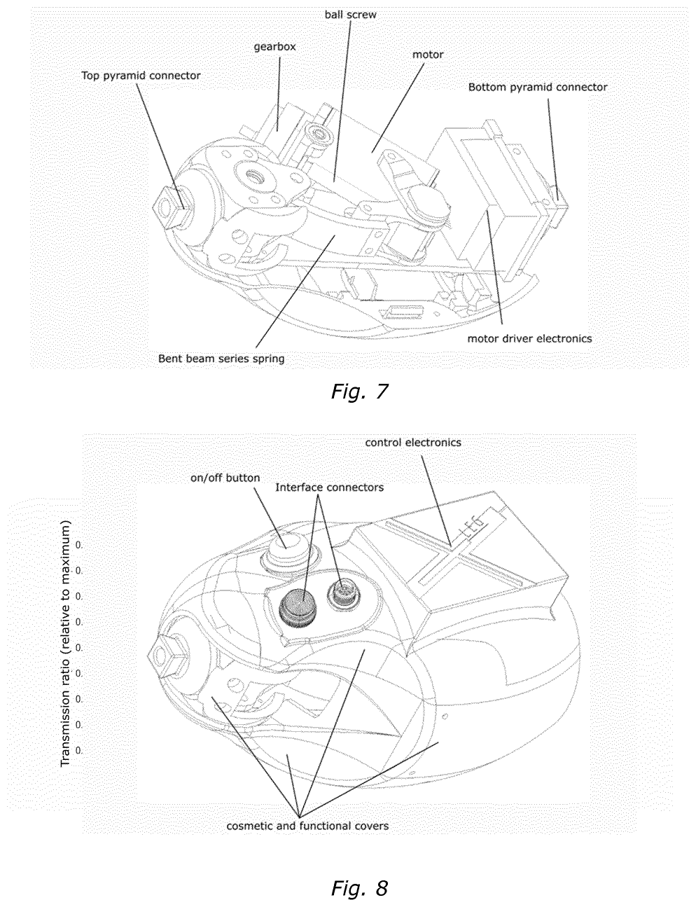

The actuation unit has been implemented in the knee prosthesis prototype as can be seen in FIG. 7-8. The Figure shows the placement of the motor and four-bar transmission with respect to the knee joint, and the entire actuation is placed centrally in the prosthesis, allowing for the placement of electronics and batteries on the sides. The entire assembly has been protected by a round 3D-printed plastic cover, allowing for a safe interaction with test subjects both avoiding crushing hazards in between mechanical components and contact with hot electronics.

The prosthesis electronics consist of two main custom electronic boards: one main board that provides an interface for all the digital and analog inputs as well as with the battery system, and embeds a National Instruments (NI) System On Module (SOM SbRIO-9651) and a nine-axis IMU (LSM9DS1 ST MicroElectronics©). The second electronics board embeds an ELMO® motor driver (Gold Twitter 15/100EE) and interfaces the control board with the Maxon motor.

The control algorithm can be divided into two main levels: the low and the high level. For the low-level control, a current controller has been implemented on the ELMO motor driver, and through the SOM either a direct current signal can be applied or the control can be done through a velocity and position feedback loop. For the high-level control, two simple state machine algorithms have been implemented, one for the walking controller and one for the sit-to-stand task. These high level algorithms are threshold based, using the sensor readings of the prosthesis encoders as well as the IMU that has been integrated into the prosthesis control electronic board.

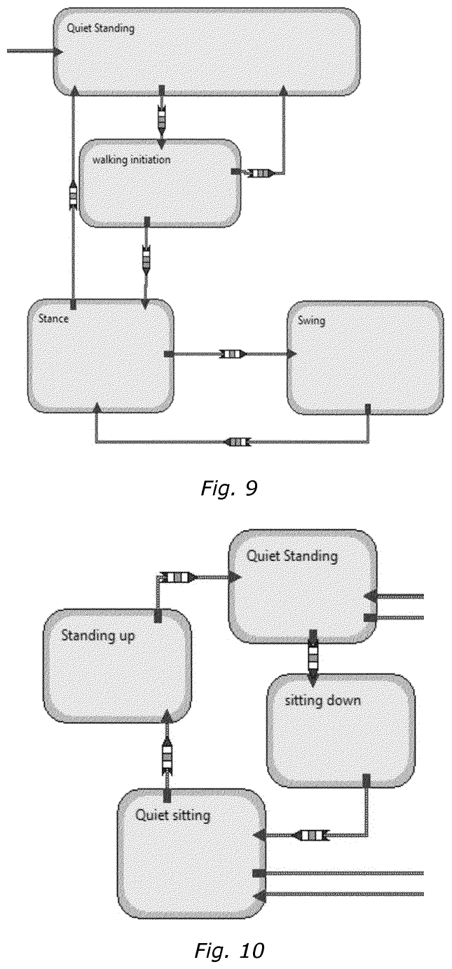

The walking state chart shown in FIG. 9 consists of four main states: a quiet standing standing state, a state representing walking initiation and two states for steady state walking, being stance and swing phases. The prosthesis default state is the quiet standing phase, and walking initiation is automatically triggered if the system detects a first step, determined based on prosthesis acceleration and velocity thresholds. For safety reasons (to avoid buckling of the prosthesis while being in stance phase), the steady state walking is only initiated when the second step is detected, subsequently the prosthesis switches between stance and swing phases, again based on acceleration and velocity thresholds. Termination of the steady state walking again triggers the quiet standing phase. During the steady state walking states, the prosthesis is controlled in position mode, setting the desired position for both swing and stance phases. As shown in FIG. 9, the default state is Quiet Standing, and through the walking initiation state the prosthesis can go into steady state walking, switching between the stance and swing phases.

The sit-to-stand statechart shown in FIG. 10 also consists of four main states. The sit-to-stand task can be initiated both in quiet standing as in quiet sitting, and the two remaining states allow to switch between those two, either by standing up or sitting down. In this case, the transitions are triggered based on knee encoder thresholds. For these tasks, the prosthesis is controlled in current mode, so applying a current value depending on the state, which automatically provides higher torques when sitting down then when standing up thanks to the implemented non-linear transmission. As shown in FIG. 10, the task can be started either in the Quiet standing or Quiet sitting phase, and there are two other states to switch between these two: the standing up and sitting down phases.

As the current value is proportionate to the amplitude of the torque profile, a fixed current value can be used for each state, making the controller very simple and robust. The current mode also allows for a transparent use, as the actuator does not need to be backdriven at any moment. While at this point only kinematic information is used as input for the control algorithm, implementing a force measurement can further improve the accuracy but more importantly the volitionality of the control, which can improve user acceptance and embodiment.

The prosthesis was validated in a bench test setup to verify the torque, velocity and transmission profiles. Static tests were performed by blocking the output and controlling the motor in current mode, investigating the relation between motor torque and output torque. Also the relation between the motor and output velocities was considered to verify the behavior of the actuator.

For the evaluation of the prosthetic prototype, a pilot study has been performed using one test subject (female, 52 years, right amputee, body weight: 54 kg, body: 162 cm). The protocol included a two-minute treadmill walk test and a sit-to-stand test. Two conditions were compared; with the X-Leg knee prosthesis and the subject's own prosthesis (MAUCH, Ossur, Iceland) in combination with a passive ankle prosthesis. Physiotherapists ensured a good alignment and fit of the novel prosthesis with respect to the subject's characteristics. During the sit-to-stand test, the subject was asked to stand up from a chair and return to the seated position (hips and knees 90°, and feet flat on the ground approximately hip distance apart). Initial hip and knee angles were verified with a goniometer. Each foot was in contact with a separate force plate. The arms were crossed in front of the chest and a metronome (set at 5 bpm) indicated the pace of the task. Two sets of ten repetitions were completed, with two minutes rest in between each set. The experimental protocol was approved by the institution medical ethics committee of UZ Brussel and Vrije Universiteit Brussel (Belgium, B.U.N. 14320194170) and by the Federal Agency for Medicines and Health Products (reference number: 80M0817). The subject was provided written and verbal information about the experimental protocol, potential risks and benefits before giving informed consent to participate in the study.

Muscle activity was continuously recorded at 1000 HZ with an electromyography (EMG) device (Mini Wave®, Cometa, Italy) for both the walking and sit-to-stand test. To assure a good EMG signal, the SENIAM guidelines were strictly followed [21]. Preparation of the skin involved shaving the hair on the place where the electrode will be placed, abrasion with sandpaper and cleaning the skin with alcohol. A maximal voluntary isometric contraction (five seconds hold for three repetitions) was included preceding the experiment. This allows expressing muscle activity relatively to a standardized measure. EMG sensors were placed on non-amputated (NAMP) side of the m. tibialis anterior (TA), m. vastus lateralis (VL), m. soleus (SOL), m. gastrocnemius medial head (GAS), m. vastus medialis (VM), m. rectus femoris (RF), m. biceps femoris caput longum (BF), m. adductor longus (ADD) and m. gluteus maximus as well as the m. gluteus maximus on the amputated side (AMP). Solely during the sit-to-stand experiment, the ground reaction forces (GRF) were measured under each foot by means of the force plates of ab instrumented split-belt treadmill (Motek Forcelink). EMG signals were pre-processed with a low and high pass filter at 500 and 20 Hz, respectively. Post-processing was executed by a third order Butterworth filter. Signals were rectified and root-mean squared in order to determine the amplitude and peak of the maximal voluntary contraction and the percentage of muscle activity during the different tasks. An amplitude of more than 10% of the maximal voluntary contraction was considered as a muscle activation. We calculated integrated EMG for each muscle as the integral of the processed signal.

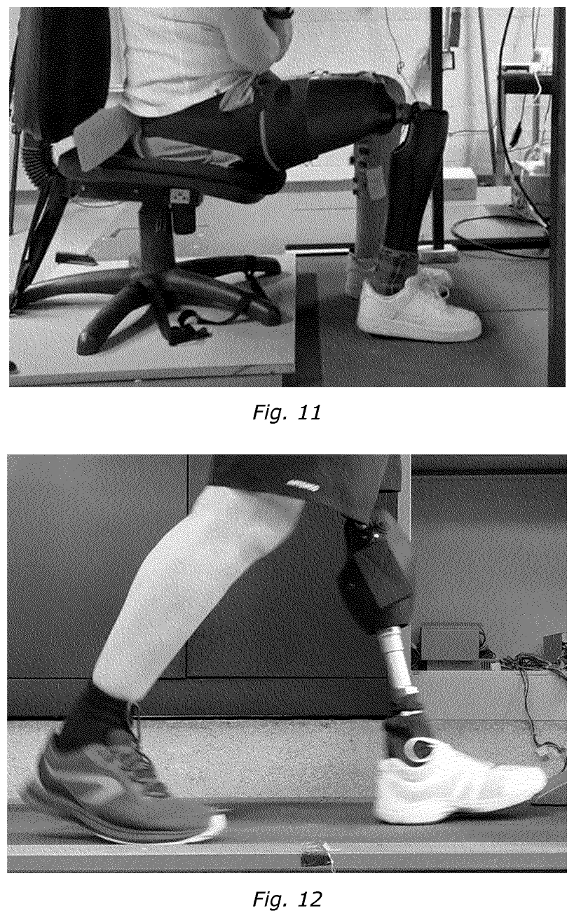

As shown in FIG. 11, the amputee tested the novel knee prosthesis mounted on top of a passive ankle prosthesis, and showed a fluent and natural walking pattern.

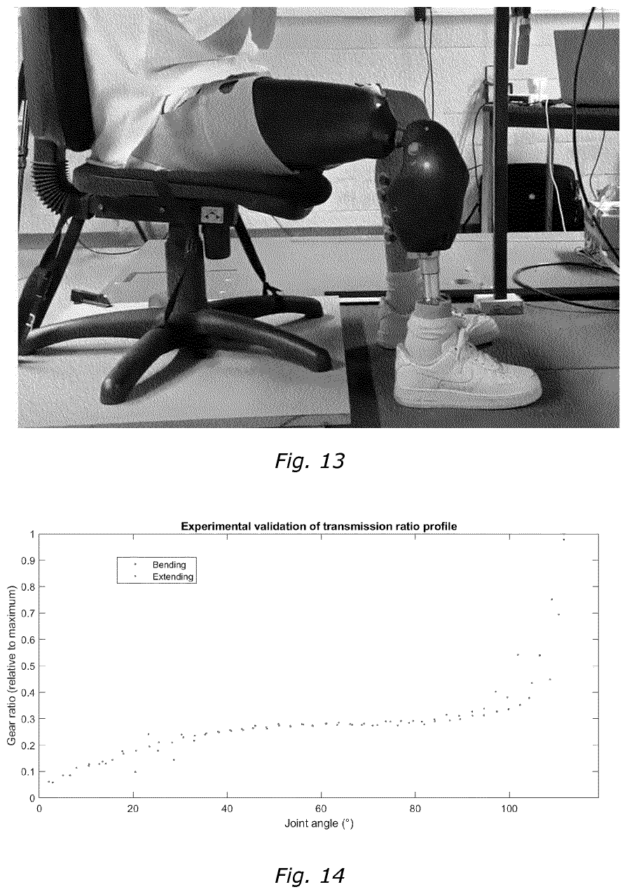

As shown in FIG. 12-13, the subject sits on a chair with a fixed height and placed the feet on the force platforms of the instrumented treadmill. Guided by a metronome sound the amputee stands up and sits down ten times.

The results of the first bench test show the expected transmission ratio between the motor and the knee joint, as can be seen in FIG. 14. The relation between motor velocity and knee velocity has been observed in a no-torque condition, resulting in the transmission ratios reported in the graph. As shown in FIG. 14, the graph shows a low transmission ratio (high output velocity) at low knee angles and a high transmission ratio (high output torque) at high knee angles.

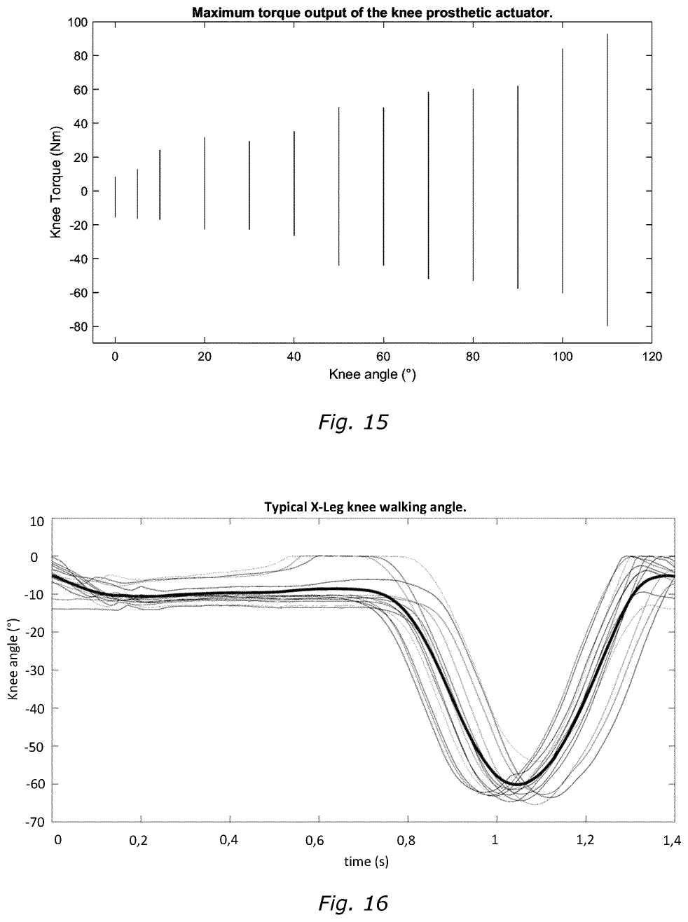

The results of a second bench test also show the transmission ratio, but by investigating the relation between the motor torque and the knee joint torque in a static experiment with a blocked output. A sinusoidal current profile is applied at the motor and the torque at the output was measured with the external torque cell. The maximum torques that can be applied are determined with this method, based on a maximum motor current of 15 A, corresponding to a motor torque of 200 mNm. The results can be seen in FIG. 15, where the maximum torque reaches +80 Nm when completely bent. As shown in FIG. 15, the maximum torque has been determined in a static test bench and corresponds to a motor torque of 200 mNm. The maximum torque that can be applied in bent conditions is above 80 Nm.

Walking experiment: the results show that the prosthesis can allow an amputee to walk with a reliable gait and provide sufficient ground clearance (FIG. 16). As shown in FIG. 16, the graph shows a repetitive behavior and high ground clearance.

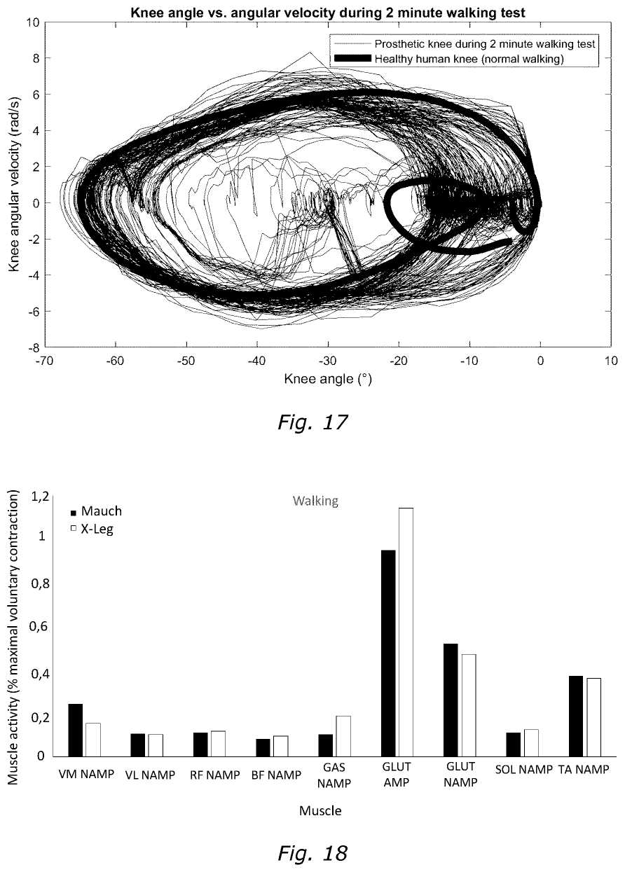

Besides this, when comparing knee joint velocities and at what angle they occur during healthy walking at a normal pace, it can be seen that the behavior of the prosthesis corresponds to what is expected from a healthy knee, with the exception of the stance phase behavior (FIG. 17). The amputee performed the walking experiment at 2.5 km/h as a self-selected walking velocity, which is slower than what is considered ‘normal walking velocity’ in literature. The maximum motor velocity required to provide the selected walking profile was 13000 rpm, which corresponds to half of the maximum achievable motor velocity in the prosthesis. As shown in FIG. 17, the thick (i.e., bold) curve shows a healthy knee gait cycle when comparing angles and angular velocities, as measured by [T. Chin et al., Comparison of different microprocessor . . . . Prosthetics and orthotics international, vol. 30, no. 1, pp. 73-80, 2006]. The blue curve shows the same comparison of the prosthetic knee during the 2-minute walking test, consisting of about 140 consecutive strides.

Decreased muscle activity was observed for the VM (−36%), VL (−4%), GLUT NAMP (−10%) and TA (−3%) during walking with the X-Leg knee prosthesis compared to the current prosthesis. Increased muscle amplitude while walking with the X-Leg knee was reported for the RF (+5%), BF (+20%), GAS (+83%), GLUT AMP (+16%) and SOL (+13%).

Sit-to-stand experiment: sit-to-stand experiments show a favorable behavior of the active prosthesis compared to the own prosthesis, resulting in lower GRF and a decrease in muscle activity.

Analysis of GRF results shows that the average force exerted by the healthy leg over one standing up cycle is 321N, while that of the prosthetic leg is only 95N when wearing the own prosthesis. When wearing the XLEG prosthesis, we see an average force of 324N exerted by the healthy leg, which is a similar number, but the force exerted by the XLEG prosthesis increased to 134N. Still a big asymmetry can be seen comparing healthy and amputated leg, but the GRF show a better distribution of the forces.

A decrease in muscle activity was observed for the knee extensors (VM: −9%; VL: −11%; RF: −17%), ankle flexors (GAS: −11%; SOL: −22%) and hip extensors (GLUT NAMP: −13%; GLUT AMP: −15%) during the sit-to-stand test with the X-Leg knee compared to the current prosthesis. A slightly increase in muscle amplitude while performing the test with the X-Leg knee was reported for the ankle extensor (TA: +2%) and knee flexor (BF: +1%).

The results of the pilot study first of all confirm the feasibility of the non-linear transmission by means of a four-bar mechanism. The results show that the prosthesis can provide rotational velocities that exceed those of normal-paced healthy walking, and can provide significant torques to assist during demanding tasks such as getting up from a chair. This can be done in a meaningful way, decreasing the required muscle activity of the amputee test subject. It can be noted that these results (only slight increases for walking capabilities and improvement for sit-to-stand) can be achieved without extensive training sessions with the amputee. The limited increases in NAMP muscle activity could be explained by the lack of training a slight increase in weight of the prosthesis—even if it is only a few 100 grams—and the fact that the walking controller was very simple. It is promising that there is a decrease in GLUT NAMP activity (as well as other muscles), as one of it's purposes is pelvis stabilization. It would make sense that assisting during flexion and extension of the prosthesis decrease the need for big compensation from the pelvis.

Looking at the capabilities of the prosthesis in terms of velocity and torque, we can say that it would be very difficult to implement this in a prosthesis without the designed non-linear transmission or another way of changing the transmission ratio. The device can output rotational velocities of 12 rad/s during walking and provide more than 80 Nm during standing up, which in the case of a constant transmission ratio would require a motor power of around 1 KW without taking into account losses or efficiencies.

There are several options to optimize this walking controller that could further increase the performance, such as automatic adaptation to velocity as well as admittance control rather than a simple position control, as well as including ground reaction forces measurements to be able to distinguish between the stance and swing phases in a more reliable way.

Major improvements could be realized by implementing a force measurement. This can improve the reactivity of the high-level controller, as the forces can be measured before there is any motion to use as input for the threshold-based controller that is used now. This way, the torque applied at the knee joint can be proportionate to the amount of downward force that is applied by the hip of the amputee for example. The torque control of the device can also be further improved by implementing a model-based controller using both the on-board angular encoders and a model of the mechanism and the elasticity that is included in it. The accuracy of this torque controller will be higher than the one that is implemented now using a current controller on the motor without having any torque feedback.

Not having stance flex capabilities in the prosthesis can be considered to be a disadvantage, however this was not reported during this pilot study. It is also not likely to be reported by amputee test subjects as they are generally accustomed to walking without the stance flex in their own prosthesis, by overextending their knee joint. Right now, as a disadvantage of the low transmission ratio the motor cannot support the high stance flex torques as they occur in a healthy person during early stance phase, even though the holding torque can be higher than what the motor itself can provide. It needs to be noted that also the four-bar mechanism and series elasticity used could relatively easily be expanded to allow for a stance flex, by tuning the series spring to have the desired stance flex stiffness and adding a secondary actuator/locking mechanism. This locking mechanism could be positioned in the four-bar mechanism so the requirements are not too high, thanks to the singular positions. The author's chose not to implement this in this prosthetic actuator given the minor importance based on the experience of testing other prosthesis prototypes, and to keep the weight and size as small as possible.

The weight of our prosthetic module with 2.3 kg is considerably lower than that of other, even commercial devices such as the Ossur Power knee (3.2 kg). The X-Leg knee is of course not a commercial device yet so the comparison is not 100% valid as it would require modifications to increase robustness and such. The prototype however did undergo a certification procedure verifying its safety and many technical checks, and has electronics on-board for interfacing with other CYBERLEGs++ modules and sensors which are not required for the operation of the prosthesis itself. This shows that a significant weight advantage can be achieved over existing products.

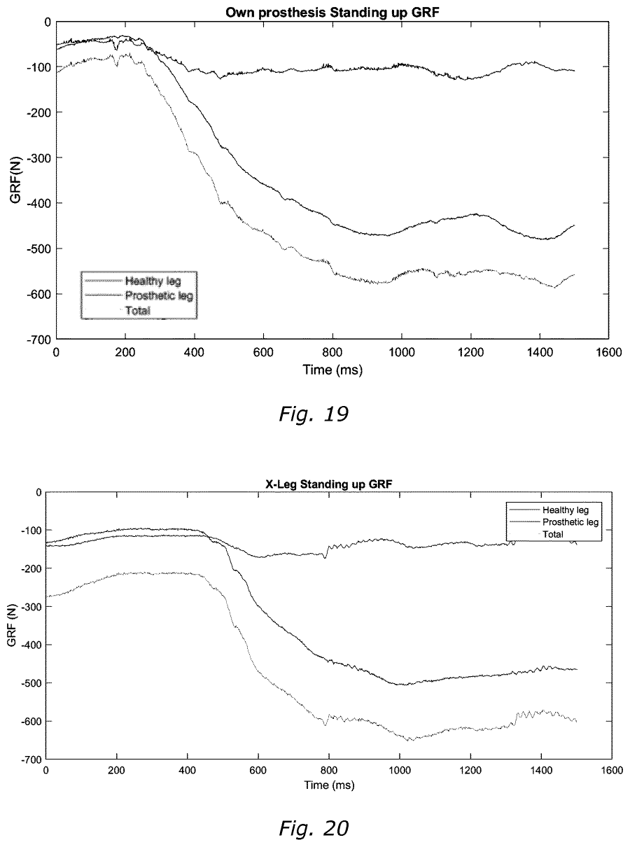

As shown in FIG. 19, there is a large asymmetry in GRF between both legs, indicating that the prosthetic side is underused. In the beginning of the movement, around 200 ms, the GRF on both sides becomes almost 0, indicating that the test subject prepares to lunge out of the chair. The average forces are 95N and 321N for the prosthetic and healthy side respectively.

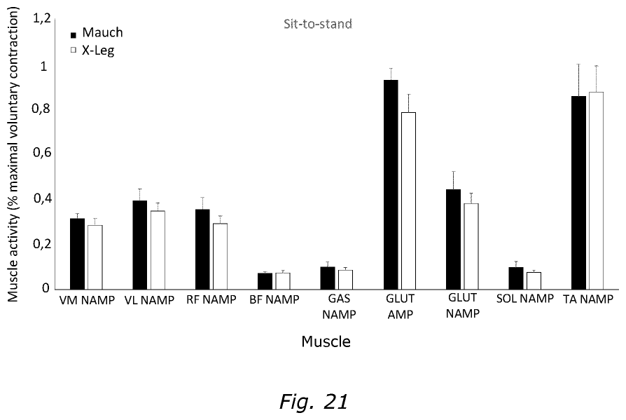

As shown in FIG. 20, however smaller than with the own prosthesis, there is still an important difference between both legs. The decrease in force in the beginning of the movement (around 200 ms) is less than with the own prosthesis, which can be explained by a decreased need to lunge and use the body weight to propel out of the chair. The average forces are 134N and 324N for the prosthetic and healthy side, respectively. As shown in FIG. 21, the difference in muscle activity (% of maximum voluntary contraction) between the current prosthesis (MAUCH) and novel prosthesis (X-Leg knee) is displayed.

The CYBERLEGs X-leg knee prosthesis is an active device that includes a novel way of actuation which has the potential of solving some of the existing problems in lower limb prosthetics. By using only a low motor power, the prosthesis can accommodate high walking velocities and provide a significant torque to assist during tasks that require this high torque. The knee prosthesis can be optimized and fine-tuned, but it already is an advanced, self-contained device that weighs barely 2.3 kg and has been proven to be useful during walking and a sit-to-stand task. By focusing on these tasks, it aims help low mobility amputees in the tasks they struggle with, potentially increasing their activity level eventually.

It is supposed that the present invention is not restricted to any form of realization described previously and that some modifications can be added to the presented example of fabrication without reappraisal of the appended claims. For example, the present invention has been described referring to a prosthetic device, but it is clear that the invention can be applied to an exoskeleton device for instance or to a orthosis device.

It is clear that the method according to the invention, and its applications, are not limited to the presented examples.

The present invention is in no way limited to the embodiments described in the examples and/or shown in the figures. On the contrary, methods according to the present invention may be realized in many different ways without departing from the scope of the invention.

1. A device for replacing or augmenting a limb of a user, comprising:
a joint,
an actuator linkage,
a base structure,
a knee structure, rotatably attached to the base structure via the joint, and a linkage mechanism comprising at least three linking elements,
wherein a first end of the linkage mechanism is coupled to the joint, a second end of the linkage mechanism is coupled to the actuator linkage, and a third end of the linkage mechanism is coupled to the base structure,
wherein the actuator linkage applies a torque to the joint through the linkage mechanism;
characterized in that the actuator linkage comprises a linear actuator with a nut and a motor to actuate the linear actuator,
wherein the actuator linkage connects a motor joint and a nut joint;
wherein a first of the linking elements is a push/pull link and connects the nut joint and a knee lever arm joint, said push/pull link comprising a structural flexible element and being elastic, wherein the knee lever arm joint is positioned on the knee structure;
wherein a second of the linking elements connects the nut joint to the base structure;
and wherein a third of the linking elements consists of a lever arm link, which connects the knee joint and the knee lever arm joint.

2. The device according to claim 1, wherein the nut is a ball-screw nut.

3. The device according to claim 1, wherein the actuator linkage applies the torque by driving at least one of the linking elements.

4. The device according to claim 1, wherein said linkage mechanism is rotationally coupled to the joint and the actuator linkage.

5. The device according to claim 1, wherein the linkage mechanism comprises at least three linking elements.

6. The device according to claim 1, further comprising a crank member having a first end connected to the actuator linkage, and a second end connected to the linkage mechanism.

7. The device according to claim 1, wherein the joint has an axis of rotation.

8. The device according to claim 1, wherein the nut is movable along the actuator linkage free from the base structure.

9. The device according to claim 1, wherein the linear actuator is a roller screw linear actuator and the nut is a roller screw nut.

10. The device according to claim 1, wherein the linear actuator is a lead screw linear actuator and the nut is a lead screw nut.

11. The device according to claim 1, wherein the linear actuator comprises a linear motor that directly moves the nut.

12. The device according to claim 1, wherein the device is a prosthesis and/or an orthosis device.

13. The device according to claim 12, wherein the device is an upper-limb prosthesis and/or orthosis, a lower-limb prosthesis and/or orthosis, or a knee prosthesis and/or orthosis.
